(19) United States
(12) Patent Application Publication
(10) Pub. No.: US 2021/0040440 A1
(43) Pub. Date: Feb. 11, 2021

(54) AN IMPROVED METHOD FOR SELECTING MICROORGANISMS

(71) Applicant: White Dog Labs, Inc., New Castle, DE (US)

(72) Inventors: Bryan P. TRACY, Wilmington, DE (US); Shawn William JONES, Bear, DE (US); Alon KARPOL, Tel-Mond (IL); Hadar GILARY, Magal (IL); Aharon M. EYAL, Jerusalem (IL)

(21) Appl. No.: 16/967,834

(22) PCT Filed: Feb. 6, 2019
(86) PCT No.: PCT/US2019/016865

§ 371 Date: Aug. 6, 2020

Related U.S. Application Data
(60) Provisional application No. 62/627,443, filed on Feb. 7, 2018.

(51) Int. Cl. C12N 1/20; C12N 1/32

(52) U.S. Cl. CPC C12N 1/20; C12N 1/32

(57) ABSTRACT

A selection method including providing a microorganism preparation including a variety of microorganisms and optionally water; providing an organic liquid including at least 70% by weight hydrophobic solvent; forming a multiple phase medium including a selected amount of the microorganism preparation and a selected amount of the organic liquid; and maintaining the multiple phase medium at a selected temperature for selected duration, whereby a treated microorganism preparation is formed.

### CROSS-RELATED REFERENCE TO RELATED APPLICATIONS

This application is a PCT International Application, which claims priority to U.S. Provisional Application No. 62/627,443, filed Feb. 7, 2018, the disclosure of which is expressly incorporated by reference herein in its entirety.

### BACKGROUND OF THE INVENTION

Microorganism selection is a key step in biotechnology, pharmaceuticals and agriculture industries directed to the isolation of new and improved strains. Such selection has high economic impact due to increased production yield, better substrate utilization, and faster process of isolating new strains. Of particularly high importance for today's industry is the selection for microorganisms such as bacteria and yeasts. That is because new microorganisms can be used as novel probiotics for human and animals and in the production of pharmaceuticals.

Known methods for microorganisms selection include selection for pathogen resistance, selection for different carbon, nitrogen, and nutrients sources and selection for survival under extreme conditions. Out of those, the industry typically prefer selection for survival under extreme conditions since they provide for new strains which can withstand the unique conditions in the gastrointestinal tracks of the animal. Yet, there are known drawbacks to these methods including that those selections do not specifically select for cells under sporulated conditions but apply only to cells in vegetative states.

### SUMMARY OF THE INVENTION

According to an embodiment, provided is a selection method comprising (i) providing a microorganism preparation comprising variety of microorganisms and optionally water; (ii) providing an organic liquid comprising at least 70% wt hydrophobic solvent; (iii) forming a multiple phase medium comprising a selected amount of said microorganism preparation and a selected amount of said organic liquid; and (iv) maintaining said multiple phase medium at a selected temperature for selected duration, whereby a treated microorganism preparation is formed; wherein (a) at least a fraction of the microorganisms in said preparation is in spore form; (b) the solubility of said hydrophobic solvent in water at 25° C. is less than 50 gram per 100 gram water; (c) said multiple phase medium comprises an aqueous phase and an organic phase and the amount of said organic liquid is selected so that the organic phase to aqueous phase weight/weight ratio is greater than 0.15 and/or said microorganism preparation comprises a given weight of cell mass and the amount of said organic liquid is selected so that the organic phase to cell mass weight/weight ratio is greater than 0.04; (d) said maintaining temperature is at least 15 degrees Celsius (15° C.); (e) said maintaining temperature is less than 70° C. ; and (f) said maintaining duration is at least 1 minute. According to an embodiment said maintaining duration is less than 30 minutes.

According to an embodiment, the method further comprises separating said treated microorganism preparation from said organic liquid, wherein at least 90% of the treated microorganism is separated within less than 10 minutes.

According to an embodiment, the method further comprises transferring at least a fraction of said treated microorganism preparation onto and/or into a growth medium and incubating at a selected temperature and for a selected duration, wherein (i) said incubating temperature is at least 15 degrees Celsius, (ii) said incubating temperature is less than 70 degrees Celsius; (iii) said incubating duration is greater than 4 hours; and said incubating duration is less than 72 hours.

According to an embodiment, said multiple phase medium is agitated during said maintaining.

According to an embodiment, said microorganism preparation comprise at least one of soil samples; human, animal, or livestock fecal samples; fermentation broths; food waste streams; wastewater treatment streams and gastrointestinal samples.

According to an embodiment, said hydrophobic solvent is selected from the group consisting of chloroform, phenol, isoamyl alcohol. According to an embodiment, said hydrophobic solvent is characterized by logarithm of partition between octanol and water greater than zero.

According to an embodiment, said transferred treated microorganism preparation is put on the surface of said growth medium. According to another embodiment, said transferred treated microorganism preparation is put into the bulk of said growth medium.

According to an embodiment, said incubating is conducted in an atmosphere comprising at least one or at least two of hydrogen, carbon dioxide and carbon monoxide.

According to an embodiment, said method further comprises selecting after incubating at least one fast growing colony. According to an embodiment, said fast growing colony comprises butyric acid producers. According to an embodiment, said fast growing colony comprises clostridia. According to an embodiment, said method further comprises selecting after incubating at least one gas generating colony. According to an embodiment, said method further comprises selecting after incubating at least one colony that does not generate gas.

According to an embodiment, provided is probiotics comprising said selected microorganism. According to an embodiment, provided is a method for improving the health of a human and/or an animal comprising feeding an effective amount of said probiotics.

### BRIEF DESCRIPTION OF THE DRAWINGS

FIG. 1. Percentage of the top 4 organism classes within non-treated and treated House B filtrates.

FIG. 2. Percentage of the top 4 organism classes within treated House B molten agar filtrate.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is a selection method comprising (i) providing an microorganism preparation comprising variety of microorganisms and optionally water; (ii) providing an organic liquid comprising at least 70 wt % hydrophobic solvent; (iii) forming a multiple phase medium comprising a selected amount of said microorganism preparation and a selected amount of said organic liquid; and (iv) maintaining said multiple phase medium at a selected temperature for selected duration, whereby a treated microorganism preparation is formed; wherein (a) at least a fraction of the microorganisms in said preparation is in spore form; (b) the solubility of said hydrophobic solvent in water at 25° C. is less than 50 gram per 100 gram water; (c) said multiple phase medium comprises an aqueous phase and an organic phase and the amount of said organic liquid is selected so that the organic phase to aqueous phase weight/weight ratio is greater than 0.15 and/or said microorganism preparation comprises a given weight of cell mass and the amount of said organic liquid is selected so that the organic phase to cell mass weight/weight ratio is greater than 0.04; (d) said maintaining temperature is at least 15 degrees Celsius (15° C.); (e) said maintaining temperature is less than 70° C,; and (f) said maintaining, duration is at least 1 minute. According to an embodiment said maintaining duration is less than 30 minutes.

According to an embodiment, said method comprises providing a microorganism preparation comprising variety of microorganisms and optionally water. According to an embodiment, said microorganisms comprise at least one of bacteria, fungi, virus, archaea, protozoa, and algea. According to an embodiment, said microorganism preparation comprise at least one of soil samples; human, animal, or livestock fecal samples; fermentation broths; food waste streams; wastewater treatment streams and gastrointestinal samples. According to an embodiment, said microorganism preparation is moisture free. According to an alternative embodiment, said microorganism preparation comprises at least 10% water, at least 20%, at least 30%, at least 40%, at least 50%, at least 60% or at least 70%.

According to an embodiment, said providing said microorganism preparation comprises mixing a sample with water, e.g. sterile water, and filtering to form a filtrate. According to an embodiment, said providing said microorganism preparation comprises wetting a solid sample. According to an embodiment, said providing said microorganism preparation comprises stressing said microorganism. According to various embodiments, said stressing comprises at least one of cultivating at carbon concentration of less than 10 mM, less than 1 mM or less than 0.1 mM; cultivating at nitrogen concentration of less than 10 mM, less than 1 mM or less than 0.1 mM; cultivating at a temperature of above 300° C., of above 400° C., of above 500° C., of above 600° C., of above 700° C.; cultivating at pH of less than 6.0, less than 5.0 or less than 4.0 and cultivating at pH of more than 8.0, more than 9.0 or more than 10.0. According to an embodiment, at least a fraction of the microorganisms in said preparation is in spore form, at least 5%, at least 10%, at least 15% or at least 20%. According to an embodiment, less than 90% of the microorganisms in said preparation is in spore form, less than 80%, less than 70%, or less than 60%.

According to an embodiment, said method comprises providing an organic liquid comprising at least 70wt % hydrophobic solvent, at least 80% or at least 90%. According to an embodiment, said organic liquid comprises an emulsion. According to an embodiment, said hydrophobic solvent is selected from the group consisting of chloroform, alkanols, aldehydes, ketones, esters, ethers and combinations thereof. According to an embodiment, said hydrophobic solvent comprises chloroform, isoamyl alcohol, phenol and combinations thereof. According to an embodiment, said hydrophobic solvent is characterized by a logP value greater than 0, or greater than 0.5 (where logP is the logarithm of partition between octanol and water). According to an embodiment, said hydrophobic solvent comprises chloroform. According to an embodiment, the solubility of said hydrophobic solvent in water at 25° C. is less than 100 gr per 100 gr water, less than 80, less than 60, less than 40, less than 20 or less than 10 gr per 100 gr water.

According to an embodiment, said method further comprises forming a multiple phase medium comprising a selected amount of said microorganism preparation and a selected amount of said organic liquid. According to various embodiments, said forming multiple phase medium comprises bringing said microorganism preparation in contact with said organic liquid, mixing them together, introducing them into a blended or mixed vessel, introduction into a mixed reactor and similar means.

According to an embodiment, said multiple phase medium comprises an aqueous phase and an organic phase and the amount of said organic liquid is selected so that the organic phase to aqueous phase weight/weight ratio is greater than 0.15, greater than 0.20, greater than 0.25, greater than 0.30, greater than 0.35, greater than 0.40, greater than 0.45, or greater than 0.50. The amount of said organic liquid required to reach the specified ratio is dependent on the mutual solubility of the hydrophobic solvent and water.

According to an embodiment said microorganism preparation comprises a given weight of cell mass and the amount of said organic liquid is selected so that the organic phase to cell mass weight/weight ratio is greater than 0.04, greater than 0.06, greater than 0.08, greater than 0.1, greater than 0.15, greater than 0.20, greater than 0.25, greater than 0.30, greater than 0.35, greater than 0.40, greater than 0.45, or greater than 0.50. The amount of said organic liquid required to reach the specified ratio is dependent on microorganism cell concentration in said preparation.

According to an embodiment, said method further comprises maintaining said multiple phase medium at a selected temperature for selected duration, whereby a treated microorganism preparation is formed. According various embodiment, said maintaining temperature is at least 15 degrees Celsius (150° C.), at least 20° C., at least 25° C., or at least 30° C.; and less than 70° C., less than 65° C., less than 60° C., less than 55° C. less than 50° C., or less than 45° C. According various embodiment, said maintaining duration is at least 1 minute (1 min), at least 2 min, at least 4 min, at least 6 min, at least 8 min or at least 10 min and less than 30 min, less than 28 min, less than 26 min, less than 24 min, less than 22 min, or less than 20 min. According to an embodiment, said multiple phase medium is agitated during said maintaining.

According to an embodiment, said method further comprising separating said treated microorganism preparation from said organic liquid, wherein at least 90% of the treated microorganism is separated within less than 10 min, less than 8 min. less than 6 min, less than 4 min or less than 2 min. Any separating method is suitable, e.g. decantation, filtration and centrifugation.

According to an embodiment, the method further comprises transferring at least a fraction of said treated microorganism preparation onto and/or into a growth medium and incubating at a selected temperature and for a selected duration, wherein said incubating temperature is at least 15° C., at least 20° C., at least 25° C., or at least 30° C.; and less than 70° C., less than 65° C., less than 60° C., less than 55° C. less than 50° C., or less than 45° C. and said incubating duration is greater than 4 hours, greater than 6 hours, greater than 8 hours or greater than 10 hours and less than 8 hours, less than 75 hours, less than 70 hours, less than 65 hours or less than 60 hours.

Any form of transferring treated microorganism preparation is suitable, e.g. pipetting. According to an embodiment, said growth medium comprises a petri dish. According to an embodiment, said growth medium comprises at least one of a carbon source, a nitrogen source, an energy source, a vitamin source, trace elements and minerals. According to an embodiment, said method comprises modifying said microorganism preparation prior to said transferring to form modified treated microorganism preparation, which modifying is selected from the group consisting of filtering, washing, centrifugating, precipitating, and combinations thereof. According to an embodiment, said treated microorganism preparation or said modified treated microorganism preparation is transferred onto the surface of said growth medium. Alternatively, it is transferred into the bulk of said growth medium, e.g. a poured plate.

According to an embodiment, said incubating is conducted in an atmosphere comprising at least one or at least two of hydrogen, carbon dioxide and carbon monoxide. According to an embodiment, said atmosphere comprises at least 5%, at least 10% or at least 15% carbon dioxide, at least 5%, at least 10% or at least 15% hydrogen and/or 5%, at least 10% or at least 15% carbon monoxide.

According to an embodiment, said method further comprises selecting after incubating at least one fast growing colony. According to an embodiment, said selecting comprises detection of colonies that appears after 3 hours, after 6 hours, after 9 hours, after 12 hours, after 15 hours, after 18 hours, after 21 hours, after 24 hours. According to an embodiment, said fast growing colony comprises butyric acid producers. According to an embodiment, said fast growing colony comprises clostridia. According to an embodiment, said selected fast growing colonies comprise at least 50 unique strains of clostridia, at least 100, at least 150, at least 200 or at least 250. According to an embodiment, said method further comprises selecting after incubating at least one gas generating colony. According to an embodiment, said method further comprises selecting after incubating at least one colony that does not generate gas. According to an embodiment, said selecting comprises checking for gas bubble embedded in the growth media and in close proximity to the colony.

According to an embodiment, the method. further comprises cultivating microorganisms in said treated. microorganism preparation. According to an embodiment, the method further comprises cultivating microorganisms selected from said growth medium. According to an embodiment of the present invention, provided is probiotics comprising microorganisms produced according to said selection method. According to an embodiment of the present invention, provided is a method. for improving the health of a human and/or an animal comprising feeding an effective amount of said probiotics. According to an embodiment of the present invention, improving health comprises providing an effective amount of said probiotics in-ovo.

### EXAMPLES

### Example 1

### Treatment of Chicken Litter with 50% (v/v) Chloroform

Chicken litter (˜100 g) was collected from three active, commercial chicken houses (A, B, and C). From each sample (A, B, and C), 10 g of chicken litter was mixed with 50 mL of sterile water. Mixtures were shaken at 100 rpm for 16 hours at 15° C. Mixtures were then passed through a 25 μm filter. The filtrate was collected and saved, while the remaining solids were discarded.

To isolate spore-forming strains, 500 μL of filtrate were mixed with 500 μL of 99.8% chloroform and incubated at room temperature for 10 minutes. Mixtures were revolved end-over-end for the 10 minute incubation. Following incubation, the mixtures were allowed to phase separate at room temperature for 1 minute. The upper aqueous phase was then serial diluted with Reinforced Clostridial Medium (RCM) (10 g/L peptone, 10 g/L beef extract, 3 g/L yeast extract, 5 g/L dextrose, 5 g/L sodium chloride, 1 g/L soluble starch, 0.5 g/L cysteine HCl, 3 g/L sodium acetate, and 0.5 g/L agar) into 100° (as is), 10⁻¹, 10⁻², 10⁻³, 10⁻⁴, and 10−5. From each dilution, 100 μL was taken applied to solid RCM agar plates, and spread. Also from each dilution, 100 μL was taken, mixed with 25 mL molten RCM agar, poured into petri dishes, and allowed to gel. All plates were incubated at 37° C. for 16 hours with an atmosphere of 5% H₂, 10% CO₂, and 85% N₂.

After 16 hours of incubation, five individual colonies from each sample (A, B, and C) were selected from the plates and placed into 10 mL of liquid RCM. These cultures were incubated at 37° C. with an atmosphere of 5% H₂, 10% CO₂, and 85% N₂ for 8 hours. At this time, samples were taken from all tubes to determine metabolite production. Additionally, 16S rDNA PCR was performed on all samples to identify the genus/species. Table 1 summarizes the results.

TABLE 1
Results from treatment with 50% (v/v) chloroform

| | Produced | | |
|---|---|---|---|
| Sample | butyric acid¹ | Identified genus | Identified species |
| A6 | − | Bacillus | paralicheniformis |
| A7 | + | Clostridium | tyrobutyricum |
| A8 | + | Clostridium | argentinense |
| A9 | + | Clostridium | perfringens |
| A10 | + | Clostridium | perfringens |
| B6 | − | Bacillus | paralicheniformis |
| B7 | + | Clostridium | limosum |
| B8 | + | Clostridium | celercrescens |
| B9 | + | Clostridium | paraputrificum |
| B10 | + | Clostridium | saccharoperbutylacetonicum |
| C6 | − | Bacillus | paralicheniformis |
| C7 | − | Clostridium | celercrescens |
| C8 | + | Clostridium | perfringens |
| C9 | + | Clostridium | paraputrificum |
| C10 | + | Clostridium | perfringens |

¹Threshold for “+” was 0.1 g/L butyric acid.

As seen from Table 1, butyric acid production was highly correlated to being identified as a Clostridium strain (11/12 strain, 91.7%), and the majority of identified strains were Clostridium (12/15 strains, 80%).

### Comparative Example A

### Non-Treatment of Chicken Litter

The filtrates prepared in Example 1 were taken and serial diluted in RCM without chloroform treatment. As in Example 1, serial dilutions were plated, incubated, and colonies selected and grown in RCM. Results are shown in Table 2.

TABLE 2
Results of Comparative Example A

| | Produced | | |
|---|---|---|---|
| Sample | butyric acid¹ | Identified genus | Identified species |
| A1 | NG | — | — |
| A2 | NG | — | — |
| A3 | − | Enterococcus | hirae |
| A4 | − | Enterococcus | hirae |
| A5 | − | Enterococcus | hirae |
| B1 | NG | — | — |
| B2 | − | Enterococcus | hirae |
| B3 | − | Enterococcus | hirae |
| B4 | − | Enterococcus | hirae |
| B5 | + | Clostridium | tyrobutyricum |
| C1 | NG | — | — |
| C2 | − | Enterococcus | hirae |
| C3 | − | Enterococcus | faecalis |
| C4 | NG | — | — |
| C5 | − | Enterococcus | diestrammenae |

¹Threshold for “+” was 0.1 g/L butyric acid. “NG” is for No Growth.
In the non-treated samples, only one of the ten strains that grew was a Clostridium and produced butyric acid (10%). Therefore, the chloroform treatment highly enriched for Clostridium and butyric acid-producing strains.

### Comparative Example B

### Treatment of Chicken Litter with 3% (v/v) Chloroform

The same filtrates from Example 1 (A, B, and C) were treated with 3% chloroform (v/v). Thirty μL of 99.8% chloroform was added to 970 μL of filtrate and incubated at 37° C. for 300 minutes at 200 rpm. The phases were allowed to separate by incubating for 20 minutes at room temperature. The upper aqueous phase was then serial diluted and plated, as in Example 1. Nine colonies from each sample were selected and grown in RCM, as in Example 1. The results are summarized in Table 3.

TABLE 3
Results of Comparative Example B

| | Produced | | |
|---|---|---|---|
| Sample | butyric acid¹ | Identified genus | Identified species |
| A11 | − | Bacillus | paralicheniformis |
| A12 | + | Clostridium | punense |
| A13 | − | Bacillus | paralicheniformis |
| A14 | NG | — | — |
| A15 | − | Caproiciproducens | galactitolivorans |
| A16 | + | Clostridium | cochlearium |
| A17 | NG | — | — |
| A18 | + | Clostridium | butyricum |
| A19 | + | Clostridium | perfringens |
| B11 | + | Clostridium | argentinense |
| B12 | − | Bacillus | paralicheniformis |
| B13 | NG | — | — |
| B14 | + | Clostridium | carboxidivorans |
| B15 | − | Bacillus | paralicheniformis |
| B16 | − | Haloimpatiens | linggiaonensis |
| B17 | + | Clostridium | carboxidivorans |
| B18 | + | Clostridium | chromiireducens |
| B19 | + | Clostridium | butyricum |
| C11 | + | Clostridium | perfringens |
| C12 | − | Bacillus | paralicheniformis |
| C13 | − | Clostridium | limosum |
| C14 | + | Clostridium | perfringens |
| C15 | + | Clostridium | haemolyticum |
| C16 | NG | — | — |
| C17 | − | Bacillus | paralicheniformis |
| C18 | − | Bacillus | paralicheniformis |
| C19 | − | Bacillus | paralicheniformis |

¹Threshold for “+” was 0.1 g/L butyric acid. “NG” is for No Growth.
Of the 23 strains that grew, 12 (52%) produced butyric acid and 13 (56%) were Clostridium. The procedure followed in this comparative example is the general procedure from the literature for isolating spore-forming colonies (i.e., 3% (v/v) chloroform). Comparing the results here to those of Example 22 show that by increasing the ratio of chloroform to 50% (v/v), we are able to increase the frequency of selecting a Clostridium strain from 56% to 80%.

### Example 2

### Generating an Enriched Library with 50% (v/v) Chloroform Treatment

The filtrate prepared from House B in Example 1 was plated onto a solid RCM agar plate without treatment and incubated at 37° C. for 16 hours with an atmosphere of 5% H₂, 10% CO₂, and 85% N₂. Additionally, the House B filtrate from Example 1 was treated with 50% (v/v) chloroform, as in Example 1, plated onto a solid RCM agar plate, and incubated at 37° C. for 16 hours with an atmosphere of 5% H₂, 10% CO₂, and 85% N₂. After the 16 hour incubation, each agar plate was cut into approximately 1 cm square pieces and transferred into 100 mL of RCM liquid media. The slurries were incubated at 37° C. for 16 hours with an atmosphere of 5% H₂, 10% CO₂, and 85% N₂. After incubation, 7 mL of broth were mixed with 3 mL of 50% glycerol and stored at −80° C. to preserve the library. A 16S rDNA library was generated from each bacterial library and sequenced via next-generation sequencing.

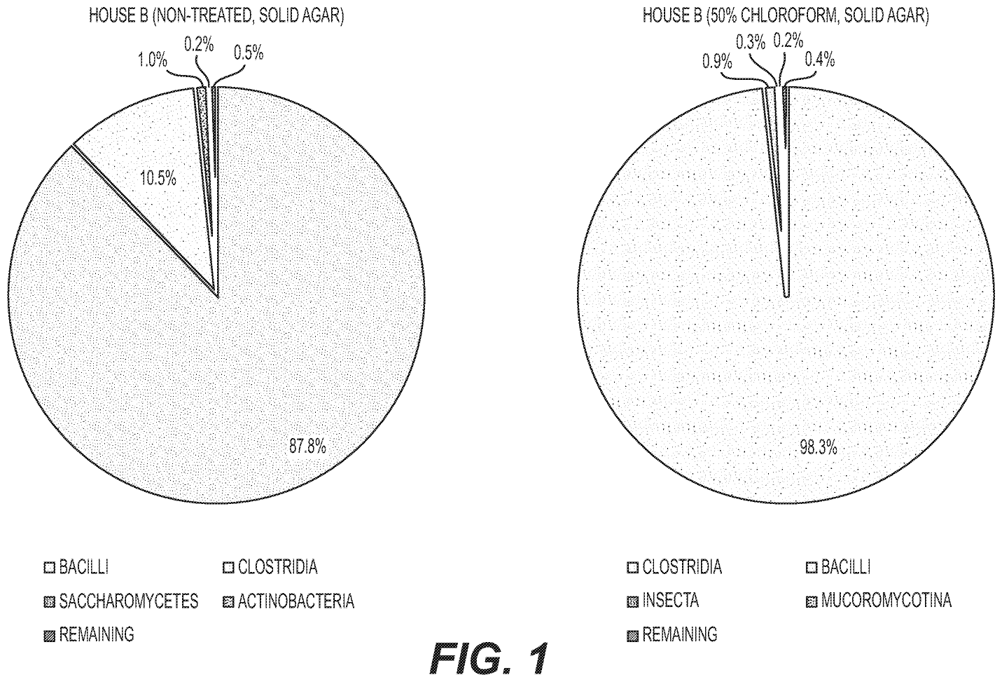

FIG. 1 shows the top 4 classes within each library (non-treated vs treated). In the non-treated sample, Bacilli make up 87.8% of all identified classes, followed by Clostridia at 10.5%, Saccharomycetes at 1.0%, and Antinobacteria at 0.2%. Following the 50% (v/v) chloroform treatment, Clostridia now make up 98.3% of the library followed by Bacilli at 0.9%, Insecta at 0.3%, and Mucoromycontina at 0.2%. Therefore, the 50o% (v/v) chloroform treatment highly enriches for Clostridia from an original 10.5% of the population up to 98. 3%. The top 10 identified strains in the treated sample are listed in Table 4.

TABLE 4
Top 10 strains identified in the 50% (v/v)
chloroform treated library from solid agar.

| Strain | % in library |
|---|---|
| Clostridium (unknown) | 31.0% |
| Clostridium butyricum | 16.3% |
| Clostridium sporogenes | 11.9% |
| Clostridium scatologenes | 9.7% |
| Clostridium sp. | 7.6% |
| Clostridium spp. | 6.8% |
| Clostridium celerecrescens | 4.0% |
| Clostridium saccharoperbutylacetonicum | 2.0% |
| Clostridium perfringens | 1.8% |
| Clostridium tyrobutyricum | 1.8% |

### Example 3

### Generating an Enriched Library with 50% (v/v) Chloroform Treatment on Molten Agar

The same 50% (v/v) chloroform treated House B filtrate from Example 1 was mixed with 25 mL molten RCM agar and then poured into a petri dish. After solidifying, the plate was incubated at 37° C. for 16 hours with an atmosphere of 5% H₂, 10% CO₂, and 85% N₂. After the 16 hour incubation, the agar plate was cut into approximately 1 cm square pieces and transferred into 100 mL of RCM liquid media. The slurry was incubated at 37° C. for 16 hours with an atmosphere of 5% H₂, 10% CO₂, and 85% N₂. After incubation, 7 mL of broth were mixed with 3 mL of 5% glycerol and stored at −80° C. to preserve the library. A 16S rDNA library was generated from the bacterial library and sequenced via next-generation sequencing.

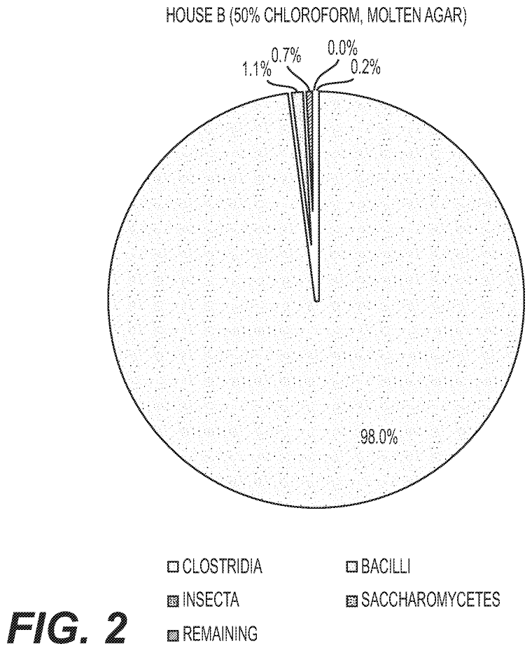

FIG. 2 shows the top 4 classes within this library. Similar to the solid agar library in Example 2, 50% (v/v) chloroform treatment highly enriched for Clostridia (98.0%). The top 10 identified strains in the treated sample are listed in Table 5.

TABLE 5
Top 10 strains identified in the 50% (v/v) chloroform
treated library from molten agar.

| Strain | % in library |
|---|---|
| Clostridium butyricum | 30.1% |
| Clostridium (unknown) | 22.0% |
| Clostridium perfringens | 18.3% |
| Clostridium sp. | 10.0% |
| Clostridium spp. | 5.9% |
| Clostridium saccharoperbutylacetonicum | 2.4% |
| Eubacterium (unknown) | 2.0% |
| Clostridium tetani | 1.6% |
| Clostridium paraputrificum | 1.0% |
| Clostridium celerecrescens | 0.7% |

Treatment of the House B filtrate with 50% (v/v) chloroform enriched for Clostridia, but the individual strains selected for differed between solid agar plating and the molten agar plating. In both libraries, C. butyricum was the most abundant identified strain, but in the solid agar library it made up 16.3% of the population, while in the molten agar library it was 30.1%, almost double. The libraries shared another five strains (Clostridium sp., Clostridium spp., C. perfringens, C. saccharoperbutylacetonicum, and C. celerecrescens) but at different abundances. Also importantly, three of the top 10 strains differed between the libraries. These differences highlight the importance of the growth medium matrix to select for different strains. Some strains prefer the flat surface of the solid agar plate while other prefer the three-dimensional support of the molten agar plate. Applying the secondary selection matrix can effect the make-up of the resulting library.

### Example 4

### Treatment of Human Fecal sample with 50% (v/v) Chloroform

Human fecal (˜10 g) was collected from a healthy male. The sample was mixed with 50mL of Tris-buffered saline. Mixture incubated for 16 hours at 15° C. Mixture was then passed through a 10 μm filter. The filtrate was collected and saved, while the remaining solids were discarded.

To isolate spore-forming strains, 500 μL of filtrate were mixed with 500 μL of 99.8% chloroform and incubated at room temperature for 10 minutes. As a control, 500 μL of filtrate without any pretreatment was prepared as well. Mixtures were revolved end-over-end for the 10-minute incubation. Following incubation, the mixtures were allowed to phase separate at room temperature for 1 minute. The upper aqueous phase was then serial diluted with Reinforced Clostridial Medium (RCM) (10 g/L peptone, 10 g/L beef extract, 3 g/L yeast extract, 5 g/L dextrose, 5 g/L sodium chloride, 1 g/L soluble starch, 0.5 g/L cysteine HCl, 3 g/L sodium acetate, and 0.5 g/L agar) into 10° (as is), 10⁻¹, 10⁻², 10⁻³, 10⁻⁴, and 10⁻⁵. From each dilution, 100 82 L was taken applied to solid RCM agar plates, and spread. All plates were incubated at 37° C. for 16 hours with an atmosphere of 5% H₂, 10% CO₂, and 85% N₂.

After 16 hours of incubation, 10 individual colonies from each sample (with chloroform and without chloroform) were selected from the plates and placed into 10 mL of liquid RCM. These cultures were incubated at 37° C. with an atmosphere of 5% H₂, 10% CO₂, and 85% N₂ for 8 hours. At this time, samples were taken from all tubes to determine metabolite production. Additionally, 16S rDNA PCR was performed on all samples to identify the genus/species. Table 5 summarizes the results.

TABLE 6
Results from human fecal treatment with 50% (v/v) chloroform

| | | | |
|---|---|---|---|
| | Produced | | |
| Sample | butyric acid¹ | Identified Class | Identified species |

Pretreated samples with 50% chloroform

| | | | |
|---|---|---|---|
| 1 | + | Clostridia | Uncultured bacteria |
| 2 | + | Clostridia | uncultured bacteria |
| 4 | + | Clostridia | uncultured bacteria |
| 5 | − | Clostridia | Ruminococcus bicirculans |
| 6 | + | Clostridia | Clostridium orbiscindens |
| 7 | − | Clostridia | uncultured bacteria |
| 8 | − | Clostridia | uncultured bacteria |
| 9 | + | Clostridia | Clostridium sp. CS1 |
| 10 | + | Clostridia | Clostridium sp. CS1 |

Control samples (w/o chloroform pretreatment)

| | | | |
|---|---|---|---|
| 12 | − | Bacteroidia | uncultured bacteria |
| 13 | − | Bacteroidia | Collinsella aerofaciens |
| 15 | − | Bacteroidia | Bacteroides vulgatus |
| 16 | − | Bacteroidia | Uncultured bacteria |
| 19 | − | Bacteroidia | Bacteroides dorei |

¹Threshold for “+” was 0.1 g/L butyric acid.

As seen from Table 6, butyric acid production was highly correlated to being identified as a Clostridia class (9/9 strain, 100%), and 30% of the strains that were isolated were clostiridium. While the control samples isolates (without chloroform pretreatment) were only from the Bacteroidia class, and were un-able to produce butyric acid.

1. A selection method comprising
(i) providing a microorganism preparation comprising variety of microorganisms and optionally water;
(ii) providing an organic liquid comprising at least 70% by weight hydrophobic solvent;
(iii) forming a multiple phase medium comprising a selected amount of said microorganism preparation and a selected amount of said organic liquid; and
(iv) maintaining said multiple phase medium at a selected temperature for selected duration, whereby a treated microorganism preparation is formed; wherein
(a) at least a fraction of the microorganisms in said preparation is in spore form;
(b) the solubility of said hydrophobic solvent in water at 25° C. is less than 50 gram per 100 gram water;
(c) said multiple phase medium comprises an aqueous phase and an organic phase and the amount of said organic liquid is selected so that the organic phase to aqueous phase weight/weight ratio is greater than 0.15 and/or said microorganism preparation comprises a given weight of cell mass and the amount of said organic liquid is selected so that the organic phase to cell mass weight/weight ratio is greater than 0.04;
(d) said maintaining temperature is at least 15 degrees Celsius;
(e) said maintaining temperature is less than 70 degrees Celsius ; and
(f) said maintaining duration is at least 1 minutes.

2. The method of claim 1, wherein said maintaining duration is less than 30 minutes.

3. The method of claim 1, further comprising separating said treated microorganism preparation from said organic liquid., wherein at least 90% of the treated microorganism is separated within less than 10 minutes.

4. The method of claim 1, further comprising transferring at least a fraction of said treated microorganism preparation onto and/or into a growth medium and incubating at a selected temperature and for a selected duration, wherein
(a) said incubating temperature is at least 15 degrees Celsius;
(h) said incubating temperature is less than 70 degrees Celsius;
(c) said incubating duration is greater than 4 hours; and
(d) said incubating duration is less than 72 hours ;

5. The method of claim 1, wherein said multiple phase medium is agitated during said maintaining.

6. The method of claim 1, wherein said microorganism preparation comprise at least one of soil samples; human, animal, or livestock fecal samples; fermentation broths; food waste streams; wastewater treatment streams and gastrointestinal samples.

7. The method of claim 4, wherein said hydrophobic solvent is selected from the group consisting of Chloroform, phenol, isoamyl alcohol.

8. The method of claim 4, wherein said hydrophobic solvent is characterized by logarithm of partition between octanol and water greater than zero.

9. method of claim 4, wherein said transferred treated microorganism preparation is put on the surface of said growth medium.

10. The method of claim 4, wherein said transferred treated microorganism preparation is put into the bulk of said growth medium.

11. method of claim 4, where incubating is conducted in an atmosphere comprising at least one or at least two of hydrogen, carbon dioxide and carbon monoxide.

12. The method of claim 4, further comprising selecting after incubating at least one fast growing colony.

13. The method of claim 12, wherein said fast growing colony comprises butyric acid producers.

14. The method of claim 12, wherein said fast growing colony comprises clostridia.

15. The method of claim 10, further comprising selecting after incubating at least one gas generating colony.

16. The method of claim 10, further comprising selecting after incubating at least one colony that does not generate gas.

17. Probiotics comprising microorganisms produced according to the method of

18. Probiotics comprising microorganisms produced according to the method of claim 4.

19. A method for improving the health of a human and/or an animal comprising feeding an effective amount of probiotics according to claim 17.

20. A method for improving the health of a human and/or an animal comprising feeding an effective amount of probiotics according to claim 18.
